(19) United States
(12) Patent Application Publication
(10) Pub. No.: US 2015/0104493 A1
(43) Pub. Date: Apr. 16, 2015

(54) Dissolvable Strip for Treatment of Oral Thermal Burns

(71) Applicants: Robert W. McDonald, III, Austin, TX (US); Kurt Fraivillig, Austin, TX (US); Adriane Shciano, Austin, TX (US); Elizabeth Vansant, Austin, TX (US); Lance Hansen, Austin, TX (US)

(72) Inventors: Robert W. McDonald, III, Austin, TX (US); Kurt Fraivillig, Austin, TX (US); Adriane Shciano, Austin, TX (US); Elizabeth Vansant, Austin, TX (US); Lance Hansen, Austin, TX (US)

(21) Appl. No.: 14/573,420

(22) Filed: Dec. 17, 2014

Related U.S. Application Data
(63) Continuation-in-part of application No. 13/732,843, filed on Jan. 2, 2013, now abandoned.
(60) Provisional application No. 61/719,697, filed on Oct. 29, 2012.
(60) Provisional application No. 61/717,082, filed on Oct. 22, 2012.

(51) Int. Cl. A61K 9/00; A61K 31/77; A61K 31/245

(52) U.S. Cl. CPC A61K 9/006; A61K 31/245; A61K 31/77
USPC 424/439; 424/78.3

(57) ABSTRACT

A consumable film adapted to adhere and to dissolve in the oral cavity that provides a local anesthetic and therapeutic agents for the treatment of oral burns or injuries. The film is designed to instantly release benzocaine, or other types of local anesthetic or therapeutic agent, upon adhesion to the affected areas of the mouth, and will continue to release sufficient quantities for pain relief and for healing over an extended period of time.

This application is a continuation of U.S. application Ser. No. 13/732,843, filed Jan. 2, 2013, which claims priority from U.S. Prov. App. No. 61/719,697, filed Oct. 29, 2012, both of which are hereby incorporated by reference. This application also claims priority from U.S. Prov. Pat. App. 61/717,082, filed Oct. 22, 2012, which is hereby incorporated by reference.

### TECHNICAL FIELD OF THE INVENTION

The present invention is related generally to fast dissolving orally consumable films for delivering one or more pharmaceutically active agent, and more particularly to fast dissolving orally consumable films containing a therapeutic agent for the treatment of oral burns.

### BACKGROUND OF THE INVENTION

Oral burns and injuries can cause pain, irritation, and blistering depending on the severity. If the injury is an thermal burn, the severity of burns depends on (1) extent, depth, and location of burn injury, (2) age of patient, (3) etiologic agents involved, and (4) coexisting or preexisting illnesses. Burns are most commonly classified by depth, ranging from first to fourth degree. Oral scald injuries due to hot food are generally classified as first and second degree burns. First and second degree burns affect the epidermis and superficial to deep dermis, respectively, causing redness, pain, and possible blistering. The oral cavity can also experience other types of cuts or injuries that affect the epidermis.

To treat pain and irritation related to oral injuries, most people turn to home remedies, such as placing ice on the burn and drinking cold water or milk. Unfortunately, these options offer short-term relief and are rather ineffective. Depending on the burn, ice can actually cause further damage. A common treatment method for the treatment of thermal burns is the use of benzocaine or other topical anesthetics, which are available over the counter. Although effective when applied directly to nerves, application of the gel may be difficult and can have an undesired taste. Dentists may add carboxymethyl cellulose and polyethylene glycol to extend contact time of benzocaine with the injury site; this paste adheres to mucosa and resists dissolution and displacement, but patients often complain about the taste.

The recent use of poloxamers to treat injuries is known. Poloxamers are nonionic triblock copolymers composed of a central hydrophobic chain of polyoxypropylene (poly(propylene oxide)) flanked by two hydrophilic chains of polyoxyethylene. Because the lengths of the polymer blocks can be customized, many different poloxamers exist that have slightly different properties. Because of their amphiphilic structure, the polymers have surfactant properties that make them useful in industrial applications. Among other things, they can be used to increase the water solubility of hydrophobic, oily substances or otherwise increase the miscibility of two substances with different hydrophobicities. Recent studies have shown that Poloxamer 188 can prevent cell death in various situations, such as electrical burns.

Poloxamer 188 can be found in several patents invented by Raphael C. Lee. In U.S. Pat. App. Pub. No. 2007/0031955 A1, Feb. 8, 2007 titled “Compositions and Methods for Refolding of Denatured Proteins,” Lee claims the use of an aqueous solution containing poloxamer 188 to refold heat-denatured proteins. In U.S. Pat. No. 5,605,687, issued Feb. 25, 1997, titled “Methods and Compositions of a Polymer (Poloxamer) for Repair of Electrical Injury,” the use of poloxamer 188 to topically treat animal tissue that has been damaged by electrical burns is disclosed. An additional usage of Poloxamer 188 is described in “Methods and Compositions of a Polymer (Poloxamer) for Cell Repair” Lee, R. C., to U.S. Pat. No. 5,470,568, issued Nov. 28, 1995. These claims utilize the poloxamer for therapeutic purposes, but do not mention treatment of oral thermal, topical burns.

Edible thin film compositions applied to the oral cavity can be designed to deliver therapeutic agents to the oral mucosa. One such example is the Listerine PocketPaks™, such as “Dissolvable Strip for Delivery of an Agent” in U.S. Pat. App. Pub. No. 2007/0218090 A1 filed Sep. 20, 2007. Another patent titled “Dissolvable Film and Method of Manufacture”, U.S. Patent Application Pub. No. 2005/0186257 filed Aug. 25, 2005, describes use of film-forming and active ingredients on a substrate and allowing the solvent to evaporate, leaving the final film as a residue. A patent titled “Edible Film Products and Methods of Making Same,” U.S. Pat. Pub. No. 2004/096569 A1, 20 May 2004, claims related rights to having colors and shapes that are indicative of flavors along the entire body of the strip. It also claims the rights to include two layers within the body of the strip and to contain a ‘medicament’ and corresponding design indicative of the medicament on the strip.

Benzocaine is well studied compound for the treatment of pain. The use of benzocaine is known to treat first degree oral burns, and in some cases, second and third degree burns. Whether the burn comes from a hot cup of coffee or a slice of pizza, different areas inside the mouth can experience different levels of thermal burns.

There remains a need in the art to develop consumable thin films containing a therapeutic agent for the treatment of oral thermal burns that provides pain relief and/or healing agents.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps. The primary market for the prototype is the general public who experience thermal oral burns, most commonly caused by ingestion of hot foods and beverages. The invention can also apply to people who suffer from chemical or electrical burns. Chemical burns can occur orally from contact with strong acids and bases, in addition to certain medications. The severity of these burns depends on the pH, concentration, volume of the agent, and the length of contact time. Electrical burns in the mouth are common because the tongue is the most sensitive part of the body to electrical currents. These injuries are most common in small children from sucking on extension cord sockets and biting electrical cords. The product can also apply to mouth injuries, mouth cuts, and mouth irritations, such as ulcers and canker sores.

### SUMMARY OF THE INVENTION

The object of the invention is to design a burn relief dissolvable strip for pain reduction and burn healing, which is loosely based on the existing drug delivery avenue of Oral Strip Technology (OST).

In one embodiment of the present invention, there is disclosed a dissolvable strip that delivers benzocaine and poloxamer 188 to the burned area for pain relief and healing. The embodiment uses an effective amount of benzocaine and poloxamer 188 and the delivery of the dissolvable strip is done so to ensure a uniform texture and composition of the active ingredients between various film samples of the dissolvable strip. Furthermore, the polymer used exhibits sufficient tensile strength, while having flexibility over brittleness, for favorable consumer use. The thin film also needs to dissolve in contact with saliva in the mouth, a process that should occur quickly enough to provide near-immediate effectiveness, but slow enough to allow adhesion to the tissue for localized administration. This adhesion would occur through mucoadhesive interactions with mucus and saliva.

All materials used must prove non-toxic by ingestion and should also be non-irritating. It should also be stable enough to have a long shelf life, given proper storage.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 shows a flow chart showing the function of the dissolvable strip;

FIG. 2 shows pictures of the samples that were made in accordance with EXAMPLE 1 and EXAMPLE 2;

FIG. 3 shows pictures of the samples that were made in accordance with EXAMPLES 3-5;

FIG. 4 shows pictures of the samples that were made in accordance with EXAMPLE 6;

FIG. 5 shows pictures of the samples that were made in accordance with EXAMPLES 7-9;

FIG. 6 shows pictures of the samples that were made in accordance with EXAMPLES 10-11; and

FIG. 7 shows a dissolvable film in accordance with EXAMPLE 12.

### DETAILED DESCRIPTION OF PREFERRED EMBODIMENTS

In one embodiment of the present invention, there is provided a dissolvable, consumable film with therapeutic agents for the treatment of injuries to the mouth. A consumable film that uses a three layer buccal delivery strip is disclosed. These strips would be comprised of a mucoadhesive layer to hold the film onto the cheek, a blocking layer to direct the drug diffusion inward to the oral cavity, and a rate-controlling polymer to deliver the active ingredients over an extended period of time. It allows for a large surface area (and therefore more capacity for drug-loading) and would avoid flexibility and mechanical strength issues associated with applying a film to the muscular and mobile tongue. The strips would allow for the drug to diffuse into the oral cavity from the cheeks. The middle layer is required for blocking.

The term “consumable” as used herein is intended to encompass substances including edible compounds, which upon administration to a consumer, is adequately tolerated without causing undue negative effects. Consumable films are shaped and sized for administration to the oral cavity of a warm-blooded animal including humans. The films are particularly well adapted to rapidly dissolve in the mouth of the warm-blooded animal. The dissolved film adheres to the surface of the mouth, typically the roof of the mouth or the tongue, and can provide a rapid delivery system for pharmaceutically active agents.

Another embodiment of the present invention considers the application of a dissolvable film to the hard palate (anterior roof of mouth). This film would include a mucoadhesive surface to hold it in place and a rate-controlling polymer to prolong drug release. This construct avoids issues related to flexibility and mechanical strength necessary for tongue application and allows consumers to resume eating while it is in place. This embodiment is not intended to be in direct contact with burned areas of the tongue.

Another embodiment of the present invention involves the application of the thin film directly to the tongue. This film would also have a mucoadhesive surface (or properties) and delivers medication/treatment directly to the surface where it is applied. A secondary advantage of tongue application is that it is easy to apply and would allow consumers to direct the strip to any other burn areas in their mouth. Special consideration is given to enhance mechanical strength and flexibility (no sharp edges or fracturing) of the strips.

Essentially, contact of the adhered strip with saliva will cause dissolution of the delivery polymer and will allow for release of the local anesthetic and interaction of the dispersed polymer with the burned surfaces.

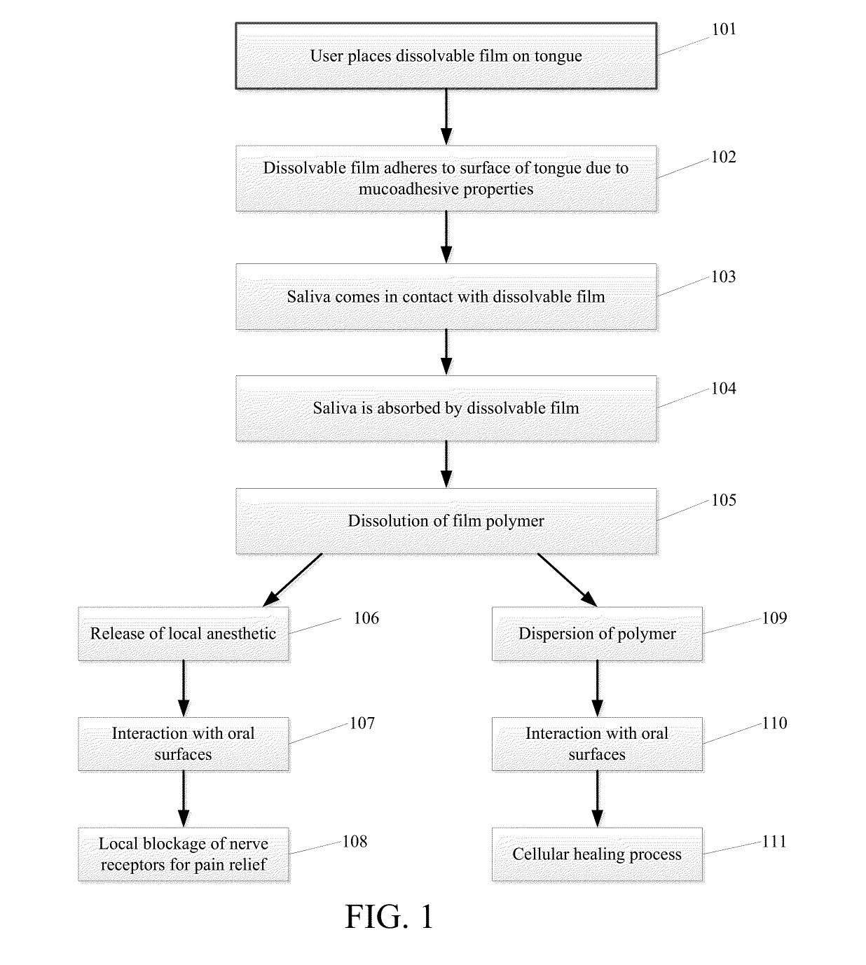

FIG. 1 shows a flow chart showing the function of the dissolvable strip. In Step 101, the user places the dissolvable film on the tongue or burn area. In Step 102, the dissolvable film adheres to the surface of the tongue or the burn area due to the mucoadhesive properties of the film. In Step 103, saliva comes in contact with the dissolvable film. In Step 104, saliva is absorbed by the dissolvable film. In Step 105, the dissolvable strip undergoes a dissolution of the polymer film. The dissolution of the film polymer performs two different function. First, it provides pain relief to the affected areas in the mouth. Second, it begins the process of cellular healing. In Step 106, the dissolution of the polymer film releases a local anesthetic. The release of the local anesthetic interacts with the affected areas in the oral cavity. The local anesthetic, in Step 107, blocks nerve receptors, which provides pain relief. The dissolution of the polymer also disperses the polymer in Step 108. The dispersion of the polymer interacts with the affected area of the oral cavity and releases an active ingredient. In Step 110, the release of an active ingredient to treat the affected areas of the mouth initiates the cellular healing process.

Benzocaine is a local anesthetic that is commonly used as a pain reliever to treat sores on body surfaces. It functions by blocking sodium from entering nerve endings and prevents the cell from depolarizing. This in turn prevents the cell from triggering an action potential and propagating the signal to the brain. By incorporating benzocaine in the present invention as the active ingredient to relieve pain, the dissolvable strip works effectively and quickly to treat the user.

The terms “active ingredient” or “pharmaceutically active agents” as used herein is intended to encompass agents other than food additives, which promote a structural and/or functional change in and/or on bodies to which they have been administered. These agents are not particularly limited, however, they should be physiologically acceptable and compatible with the film.

Lidocaine is a local anesthetic and is used to treat irritation and burns on body surfaces. Like benzocaine, it functions by blocking sodium from entering nerve cells that prevents signaling to the brain. It can also act as an active ingredient to relieve pain in the present invention.

Menthol is an organic substance that is derived from mint and can be used to treat pain and irritation. In modern medications, it is commonly used to treat minor throat irritation or soreness. Menthol functions by triggering cold-sensitive TRPM8 receptors on the area of application, creating a cooling sensation as the nerve cells propagate the signal to the brain.

Pullulan is polysaccharide polymer that is commonly used in oral strip applications, such as the Listerine breath strips. It is mostly tasteless and dissolves when it comes into contact with water or saliva. It is very frequently used in drug delivery applications because of its oxygen blocking properties, which help preserve active ingredients embedded within the polymer.

HPMC, or Hydroxypropyl methylcellulose, is a viscoelastic polymer that is also commonly used in oral delivery applications. An HPMC polymer strip is designed to release the active ingredients using particulate dispersion at a constant rate over time. The HPMC scaffold is created by dissolving the polymer in a combination of water and ethanol. The ethanol disperses the polymer while the addition of water causes the polymer to cross-link and entangle, creating a physical mesh. This matrix of HPMC is able to dissolve when it comes into contact with ethanol or salivary enzymes.

Poloxamer 188 is disclosed to be used as the scaffold for the film. This polymer has a unique property of repairing cell membranes as well as the abilities to dissolve in water and to store pharmaceuticals. Poloxamer 188 acts as a synthetic chaperone by helping refold heat-damaged proteins, returning them to high levels of functionality. They are synthesized by the sequential addition of propylene oxide and ethylene oxide to a base of a low molecular weight propylene glycol. Propylene glycol is a water-soluble organic compound that becomes water-insoluble when the molecular weight increases above 750 g/mol. The addition of ethylene oxide in the final synthesis step returns water solubility to the molecule, which is a critical characteristic for application in the mouth. Water solubility of poloxamers increases if (1) the percentage of ethylene oxide increases or (2) the molecular weight of propylene glycol decreases. Both of these methods can be used when tailoring a poloxamer.

Other water soluble polymers are considered for the present invention. The water soluble polymer used in the films of the present invention can be selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

### EXAMPLE 1

The ingredients listed in Table 1 were combined to provide a dissolvable film of the present invention. Unless specified otherwise, the term “% by weight” as used herein with reference to the final product (i.e., the film, as opposed to the formulation used to produce the film), denotes the percent of the total dry weight contributed by the subject ingredient.

TABLE 1

| Formulation | | | Theory | A | B |
|---|---|---|---|---|---|
| 1 | | % | (g) | film | film |
| Solids | Pullulan | 55 | 2.750 | | |
| | Poloxamer | 15 | 0.750 | | |
| | 188 | | | | |
| | Benzocaine | 20 | 1.000 | | |
| | Glycerol | 10 | 0.500 | | |
| Film | TOTAL | 10.0 | 5.000 | | |
| Solution | Solids | | | | |
| | Water | 90.0 | 45.000 | | |
| | TOTAL | 100.0 | 50.000 | 10 g | 20 g |
| Mixing | homogenizer | | | | |

This example shows a formulation wherein it was easily dissolvable, except benzocaine. Other suitable replacements for benzocaine is disclosed.

### EXAMPLE 2

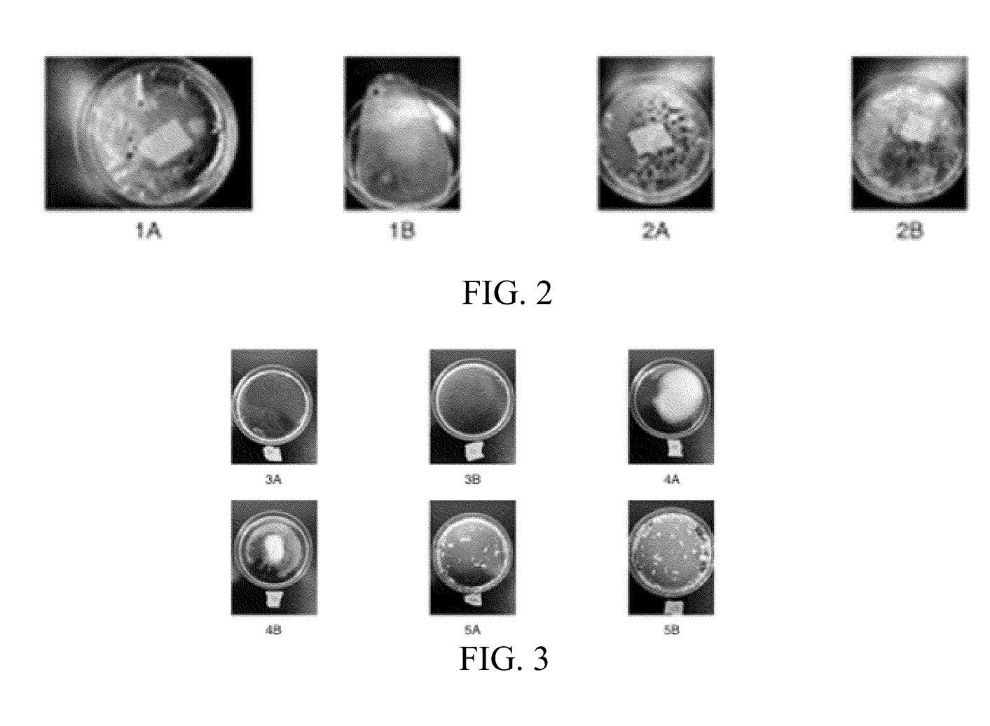

The ingredients listed in Table 2 were combined to provide a dissolvable film of the present invention. FIG. 2 shows pictures of the samples that were made in accordance with EXAMPLE 1 and EXAMPLE 2. Samples 1A and 1B of FIG. 2 are test samples made in accordance with EXAMPLE 1. Samples 2A and 2B of FIG. 2 are test samples made in accordance with EXAMPLE 2.

TABLE 2

| Formulation | | | Theory | A | B |
|---|---|---|---|---|---|
| 2 | | % | (g) | film | film |
| Solids | Pullulan | 50 | 2.500 | | |
| | Poloxamer | 15 | 0.750 | | |
| | 188 | | | | |
| | Benzocaine | 20 | 1.000 | | |
| | Glycerol | 15 | 0.750 | | |
| Film | TOTAL | 10.0 | 5.000 | | |
| Solution | Solids | | | | |
| | Water | 90.0 | 45.000 | | |
| | TOTAL | 100.0 | 50.000 | 10 g | 20 g |
| Mixing | homogenizer | | | | |

For EXAMPLES 1 and 2, the ingredients in TABLES 1 and 2 were combined to provide a consumable film in accordance with the following procedure:

The water and glycerol were mixed. Poloxamer 188 was added until dissolved. Benzocaine was next added. Pullulan was next added. The mixture was runned through a homogenizer. The films were plated and weighed.

This example shows a formulation wherein it was easily dissolvable, except benzocaine. Other suitable replacements for benzocaine is disclosed.

### EXAMPLE 3

The ingredients listed in Table 3 were combined to provide a dissolvable film of the present invention.

TABLE 3

| Formulation | | | Theory | A | B |
|---|---|---|---|---|---|
| 3 | | % | (g) | film | film |
| Solids | Pullulan | 0 | 0.000 | | |
| | Poloxamer | 65 | 3.250 | | |
| | 188 | | | | |
| | Benzocaine | 20 | 1.000 | | |
| | Glycerol | 15 | 0.750 | | |
| Film | TOTAL | 10.0 | 5.000 | | |
| Solution | Solids | | | | |
| | Ethanol | 90.0 | 45.000 | | |
| | TOTAL | 100.0 | 50.000 | 10 g | 20 g |
| Mixing | stir bar | | | | |
| Method | | | | | |

### EXAMPLE 4

The ingredients listed in Table 4 were combined to provide a dissolvable film of the present invention.

TABLE 4

| Formulation | | | Theory | A | B |
|---|---|---|---|---|---|
| 4 | | % | (g) | film | film |
| Solids | Pullulan | 45 | 2.250 | | |
| | Poloxamer | 15 | 0.750 | | |
| | 188 | | | | |
| | Benzocaine | 20 | 1.000 | | |
| | Glycerol | 20 | 1.000 | | |
| Film | TOTAL | 10.0 | 5.000 | | |
| Solution | Solids | | | | |
| | Water | 90.0 | 45.000 | | |
| | TOTAL | 100.0 | 50.000 | 10 g | 20 g |
| Mixing | mortar and | | | | |
| Method | pestle | | | | |

### EXAMPLE 5

The ingredients listed in Table 5 were combined to provide a dissolvable film of the present invention.

FIG. 3 shows pictures of the samples that were made in accordance with EXAMPLES 3-5. Samples 3A and 3B of FIG. 3 are test samples made in accordance with EXAMPLE 3. Samples 4A and 4B of FIG. 3 are test samples made in accordance with EXAMPLE 4. Samples 5A and 5B of FIG. 3 are test samples made in accordance with EXAMPLE 5. The formulation lacked uniformity of distribution of solids and texture.

With EXAMPLES 3-5, the same procedure for the mixing of the chemicals was used in accordance with EXAMPLES 1 and 2 above, except that EXAMPLES 3 and 5 were mixed in ethanol instead of water. A stir bar was used for mixing. EXAMPLE 4 was mixed with water, and a mortar and pestle was used to mix in the benzocaine.

TABLE 5

| Formulation | | | Theory | A | B |
|---|---|---|---|---|---|
| 5 | | % | (g) | film | film |
| Solids | Pullulan | 0 | 0.000 | | |
| | Poloxamer 188 | 75 | 7.500 | | |
| | Benzocaine | 10 | 1.000 | | |
| | Glycerol | 15 | 1.500 | | |
| Film | TOTAL Solids | 20.0 | 5.000 | | |
| Solution | | | | | |
| | Ethanol | 80.0 | 45.000 | | |
| | TOTAL | 100.0 | 50.000 | 10 g | 20 g |
| Mixing | stir bar | | | | |
| Method | | | | | |

### EXAMPLE 6

The ingredients listed in Table 6 were combined to provide a dissolvable film of the present invention.

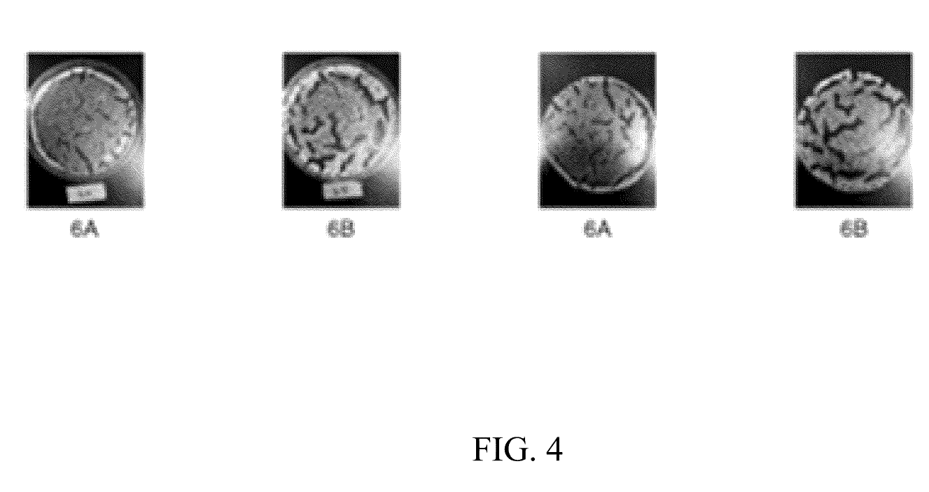

FIG. 4 shows pictures of the samples that were made in accordance with EXAMPLE 6. Samples 6A and 6B of FIG. 4 are test samples made in accordance with EXAMPLE 6.

TABLE 6

| Formulation | | | Theory | A | B |
|---|---|---|---|---|---|
| 6 | | % | (g) | film | film |
| Solids | HPMC | 63.33 | 9.500 | | |
| | Poloxamer 188 | 15.00 | 2.250 | | |
| | Benzocaine | 6.67 | 1.000 | | |
| | Glycerol | 15.00 | 2.250 | | |
| Film Solution | TOTAL Solids | 15.00 | 15.000 | | |
| | Water | 4.25 | 4.250 | | |
| | Ethanol | 80.75 | 80.750 | | |
| | TOTAL | 100.00 | 100.00 | 10 g | 20 g |
| Mixing Method | homogenizer | | | | |
| Density (g/mL) | water | 1.00 | | | |
| | ethanol | 0.79 | 51.17237009 | | |

This example shows a formulation wherein there were existence of bubbles and small channels. The film 6A proved to have good pliability and sturdiness while the film 6B had poor pliability with more brittle characteristics.

### EXAMPLE 7

The ingredients listed in Table 7 were combined to provide a dissolvable film of the present invention.

TABLE 7

| Formulation | | | Theory | A | B |
|---|---|---|---|---|---|
| 7 | | % | (g) | film | film |
| Solids | HPMC | 63.33 | 4.750 | | |
| | Poloxamer | 15.00 | 1.125 | | |
| | 188 | | | | |
| | Benzocaine | 6.67 | 0.500 | | |
| | Glycerol | 15.00 | 1.125 | | |
| Film Solution | TOTAL | 15.00 | 7.500 | | |
| | Solids | | | | |
| | Water | 4.25 | 2.125 | | |
| | Ethanol | 80.75 | 40.375 | | |
| | TOTAL | 100.00 | 50.000 | 10 g | 20 g |
| Mixing | homogenizer | | | | |
| Method | | | | | |
| Plate | PTFE | | | | |

This example had films 7A and 7B with cloudy appearance and uneven distribution of solids and textures. They were both pliable.

With EXAMPLES 6 and 7, the same type of procedure for mixing the compounds is used as described in EXAMPLES above. A homogenizer was used for the mixing method. EXAMPLE 6 used plastic plates. EXAMPLE 7 used PTFE plates. In these EXAMPLES 6 and 7, the mixing was first performed by using a measured amount of ethanol. Glycerol was then next added. Poloxamer 188 was then next added. And Benzocaine was next added followed by a low amount of heat (above room temperature). Measured amounts of HPMC was then next added. And water was then slowly added to final mixture.

### EXAMPLE 8

The ingredients listed in Table 8 were combined to provide a dissolvable film of the present invention.

TABLE 8

| Formulation | | | Theory | A |
|---|---|---|---|---|
| 8 | | % | (g) | film |
| Solids | HPMC | 78.33 | 5.875 | |
| | Poloxamer | 0.00 | 0.000 | |
| | 188 | | | |
| | Benzocaine | 6.67 | 0.500 | |
| | Glycerol | 15.00 | 1.125 | |
| Film Solution | TOTAL | 15.00 | 7.500 | |
| | Solids | | | |
| | Water | 4.25 | 2.125 | |
| | Ethanol | 80.75 | 40.375 | |
| | TOTAL | 100.00 | 50.000 | 10 g |
| Mixing | top-down impeller | | | |
| Method | | | | |
| Plate | PTFE | | | |

This example shows a formulation wherein the final film showed bubbly characteristics. The samples were non-pliable, stiff, and the examples cracked on touch. This is an example where no poloxamer 188 was used. This example of dissolvable strip would only provide pain relief from the benzocaine.

### EXAMPLE 9

The ingredients listed in Table 9 were combined to provide a dissolvable film of the present invention.

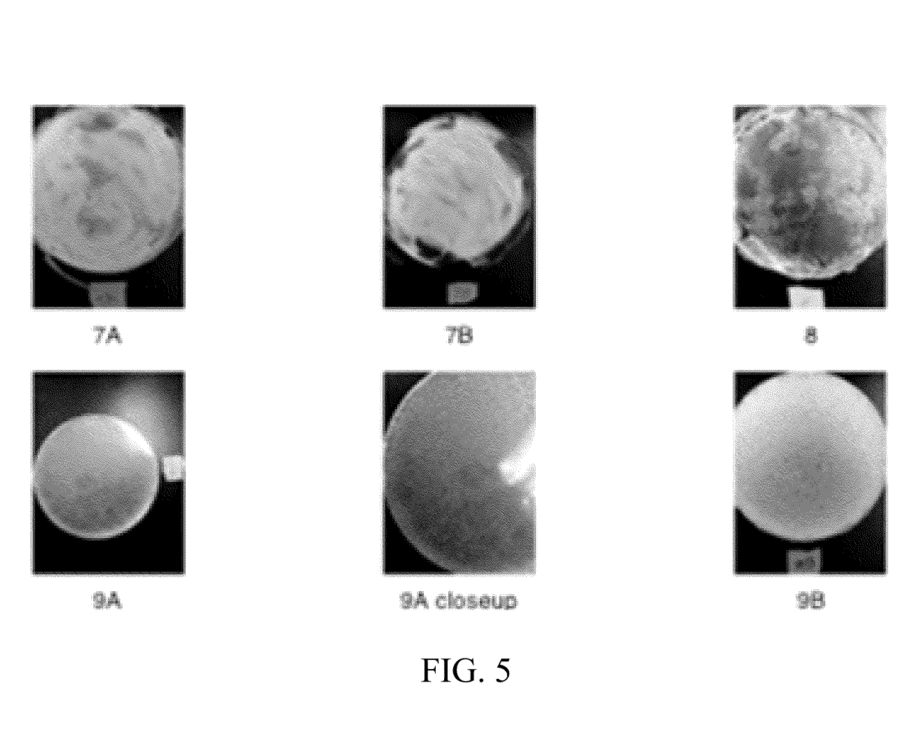

FIG. 5 shows pictures of the samples that were made in accordance with EXAMPLES 7-9. Samples 7A and 7B are test samples made in accordance with EXAMPLE 7. Samples 8A and 8B are test samples made in accordance with EXAMPLE 8. Samples 9A and 9B are test samples made in accordance with EXAMPLE 9.

TABLE 9

| Formulation | | | Theory | A | B |
|---|---|---|---|---|---|
| 9 | | % | (g) | film | film |
| Solids | HPMC | 63.33 | 4.750 | | |
| | Poloxamer | 15.00 | 1.125 | | |
| | 188 | | | | |
| | Benzocaine | 6.67 | 0.500 | | |
| | Glycerol | 15.00 | 1.125 | | |
| Film Solution | TOTAL | 15.00 | 7.500 | | |
| | Solids | | | | |
| | Water | 1.70 | 1.000 | | |
| | Ethanol | 83.30 | 41.650 | | |
| | TOTAL | 100.00 | 50.150 | 10 g | 20 g |

This example shows a formulation wherein the dissolvable films were pliable and uniform. There was some benzocaine recrystallization.

### EXAMPLE 10

The ingredients listed in Table 10 were combined to provide a dissolvable film of the present invention.

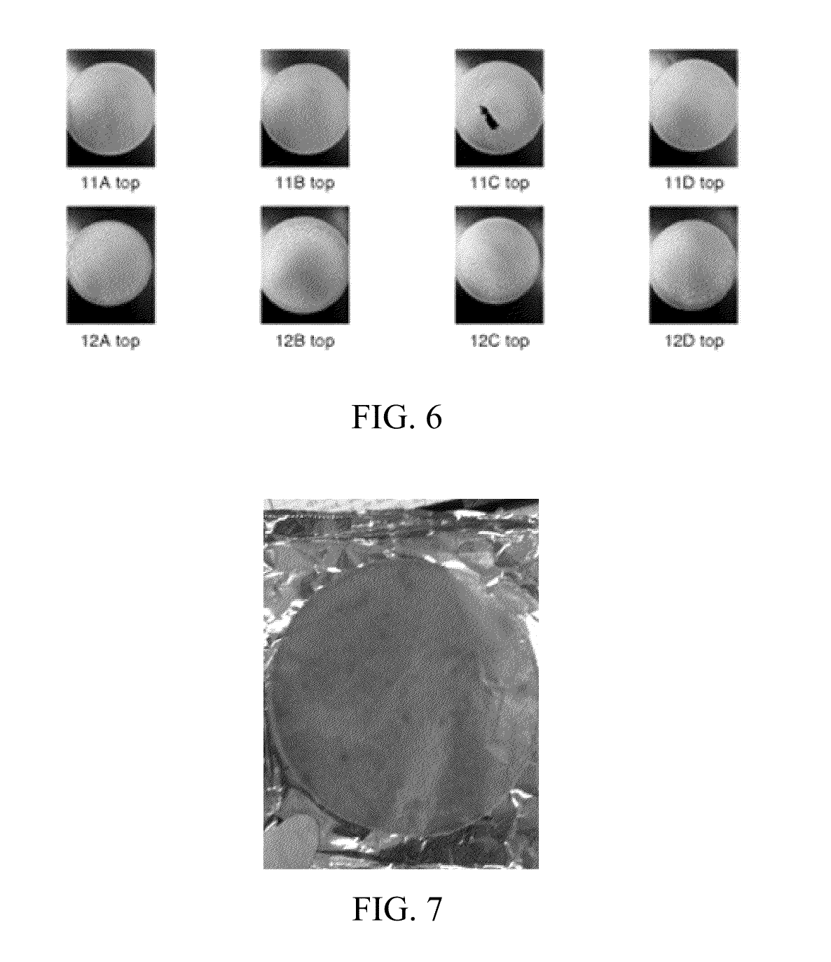

FIG. 6 shows pictures of the samples that were made in accordance with EXAMPLES 10-11. Samples 11A to 11D are test samples made in accordance with EXAMPLE 10. Samples 12A to 12D are test samples made in accordance with EXAMPLE 11.

TABLE 10

| Formulation | | | Theory | A, B, C, D |
|---|---|---|---|---|
| 10 | | % | (g) | films |
| Solids | HPMC | 55.00 | 4.125 | |
| | Poloxamer | 20.00 | 1.500 | |
| | 188 | | | |
| | Benzocaine | 10.00 | 0.750 | |
| | Glycerol | 15.00 | 1.125 | |
| Film Solution | TOTAL | 15.00 | 7.500 | |
| | Solids | | | |
| | Water | 1.70 | 0.850 | |
| | Ethanol | 83.30 | 41.650 | |
| | TOTAL | 100.00 | 50.000 | 10 g |
| Mixing | stir bar | | | |

This example shows a formulation wherein the dissolvable film was thin and very pliable. There was existence of benzocaine recrystallization.

### EXAMPLE 11

The ingredients listed in Table 11 were combined to provide a dissolvable film of the present invention.

TABLE 11

| Formulation | | | Theory | A, B, C, D |
|---|---|---|---|---|
| 11 | | % | (g) | films |
| Solids | HPMC | 55.00 | 4.125 | |
| | Poloxamer | 20.00 | 1.500 | |
| | 188 | | | |
| | Benzocaine | 10.00 | 0.750 | |
| | Glycerol | 15.00 | 1.125 | |
| Film Solution | TOTAL | 15.00 | 7.500 | |
| | Solids | | | |
| | Water | 0.00 | 0.000 | |
| | Ethanol | 85.00 | 42.500 | |
| | TOTAL | 100.00 | 50.000 | 10 g |
| Mixing | stir bar | | | |
| Method | | | | |

This example shows a formulation wherein the dissolvable films were thin and very pliable. There was existence of benzocaine recrystallization. There was slight cracking and film 12C showed poor distribution of solids.

Formulations for EXAMPLES 8-11, including the present embodiments of the current invention use the same type of mixing as discussed above. In accordance, measured amounts of ethanol was placed into a beaker using a stir bar. Other types of stirring methods are contemplated depending on the amount used. Commercial manufacture of the current embodiments would include larger scaled production and applicable stirring, mixing, and finalizing methods that is associated with larger productions. Glycerol was added and stirred. Polozmer 188 was added and stirred for approximately 3 to 5 minutes. Benzocaine was then added. Heat was then added (60 degrees C.) and mixed until the solution began to appeal clear or settled. HPMC was then slowly added. Finally, water was added in a drop-wise manner until the final solution was obtained. Strips were made in accordance with the finalized procedures for manufacture and cut into appropriate size for use and testing.

In addition to the basic layer polymers, a plasticizer in the tongue strip construct was incorporated. The plasticizer increases the flexibility of the polymer film by reducing the glass transition temperature. This inclusion softens the tongue strip as it enters the oral cavity, preventing possible user irritation. Glycerol as plasticizer was used because it is tasteless and dissolves readily in ethanol and water.

To quantify the mechanical properties of the film, a Texture Analyzer (TA) was used. This device is a measurement tool used to assess physical properties of materials, including drug delivery systems. The qualities it has the ability to measure include tensile strength, hardness, and bioadhesive properties. TA was used to measure the uniformity of tensile strength and hardness between the film samples, since these qualities relate to the consistency of film components.

To measure the tensile strength of the films, an elongation test was performed, which stretched the film outward at a constant rate until it fractured. During this time, the force exerted on the apparatus and the elongation distance was recorded. From data collected, the tensile strength was tested and the Young's modulus over different elongation lengths. A maximum elongation force was recorded before the film broke in half. Young's Moduli are displayed below.

Average Young's Moduli of strips.

Mechanical properties of films from Formulations 10 and 11 were tested. Formulation 10 used 98% ethanol and 2% water as a solvent, while Formulation 11 used 100% ethanol. Of these formulations, two different films from each were tested. 11C and 11D, and 12C and 12D of FIG. 6 are the results of these tests. The C films were poured from the top of the batch beaker, while D films were poured from the bottom of the batch beaker. C films were more uniform since many of the heavier contents of the solution probably sank to the bottom of the beaker, causing the D films to be more varied.

Full mechanical testing data is as follows:

To quantify benzocaine release, a standard curve for benzocaine in each of the solvents using a spectrophotometric plate reader was generated. A series of 10 dilutions were prepared using a factor of 0.5 and placed into a 96-well plate. Testing for benzocaine release was fairly straightforward using a basket apparatus, as described in the United States Pharmacopeia. Using the dissolution apparatus, solution samples were acquired at set time points, placed them in the 96-well plate, and read the samples. By comparing the results to the standard curve, benzocaine was quantified as a measure released over time. The benzocaine release profile of the samples is displayed below.

Benzocaine release versus time of six films from Formulation 10.

To quantify the release of benzocaine, six different strips from Formulation 10 were tested and recorded the amount of released benzocaine (milligrams) over a ten minute time period. After plotting each individual sample, the average of the benzocaine amount at each time point was calculated. An average release profile was calculated. From the linear regression of this data, R² value of 0.9974 was obtained, correlating to a very linear release profile. From the linear regression, the final prototype dispenses approximately 0.87 milligrams of benzocaine per minute.

In addition, an overnight endpoint dissolution test was performed with six strips from Formulation 11. After twenty hours in the orbital shaker, each test strip thoroughly dissolved in the PBS solution. Each sample was analyzed using the plate reader and compared to the standard curve to determine content uniformity. The results showed that approximately 75% of the total benzocaine in the final prototype is released in the first 10 minutes of dissolution.

Since poloxamer 188 did not have a detectable wavelength, the polymer with Cobalt (II) thiocyanate (CT) was complexed. CT has an absorbance wavelength at 624 nm, and when complexed with poloxamer 188, could help to quantify the amount of dissolved poloxamer at each time point. A thin, pliable, and uniform film containing all active ingredients by modifying the solvents from deionized (DI) water to an ethanol/DI water combination.

### EXAMPLE 12

The ingredients listed in Table 12 were combined to provide a dissolvable film of the present invention.

TABLE 12

| | Materials | Final % | Amount (mg) |
|---|---|---|---|
| Composition of Solids | Benzocaine | 10 | 24 |
| (Solids are 15% | Poloxamer 188 | 20 | 48 |
| of the Solution) | HPMC | 55 | 120 |
| | Glycerol | 15 | 36 |
| Composition of Solvents | Ethanol | 98 | — |
| | Water | 2 | — |

EXAMPLE 12 contains poloxamer 188, benzocaine, glycerol, HPMC, maltodextrin/sucralose, and mango flavoring in a solvent mixture of 98%/2% of Ethanol/DI Water. FIG. 7 shows a dissolvable film in accordance with EXAMPLE 12. Production strips are cut into 3 cm×4 cm rectangles. The size of this strip is ideal for the delivered dosage, as well as cost per strip analysis.

The benzocaine concentration was chosen to provide long lasting relief, while staying well under the toxicity level of the drug. Through initial testing, it was determined that 20% poloxamer was necessary to create a remaining polymer layer over the wound. The HPMC concentration is tunable, with the final additions of sweeteners and flavorings. The glycerol concentration was determined experimentally, and 15% allowed the plasticizer to provide enough flexibility without degrading the integrity of the film.

Useful sweetening agents include A) water-soluble sweetening agents such as, for example, monosaccharides, disaccharides and polysaccharides, B) water-soluble artificial sweetening agents such as, for example, soluble saccharin salts and the like, C) dipeptide based sweetening agents such as L-aspartic acid derived sweetening agents and the like, D) protein based sweeteners such as, for example, thaumatoccous danielli (Thaumatin I and II), and mixtures thereof. Additional suitable sweeteners include sucralose, aspartame, acesulfame potassium, neotame, saccharin, xylitol and mixtures thereof.

Other additions to the film strip, such as maltodextrin/sucralose, mango flavoring, and food coloring can be added.

Because the median lethal doses, or LD₅₀ values, for poloxamers are very high (greater than 5 g/kg), poloxamer 188 is present in less than 50-milligram concentrations. The LD₅₀ for benzocaine is between 0.5 and 5 g/kg, and benzocaine in the current embodiments is provided in less than 25-milligram concentrations for each strip. The additional main materials incorporated into the design include Hydroxypropyl methylcellulose (HPMC) and glycerol.

Other additions to embodiment included maltodextrin/sucralose, flavoring, and food grade coloring. Each addition, present in small amounts, is used commonly in other oral strips on the market and presents negligible toxicity concern.

Other types of compounds are considered for the polymer base of the dissolvable strip. They include pullulan, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

An initial concern is to ensure that the PTFE trays used for setting films are perfectly level and free of any markings on the exposed surface. Since the benzocaine is mainly dispersed in particle form and not completely dissolved, any unevenness will cause a large migration of the heavier drug crystals. When mixing the ingredients into the solvent, stirring must occur at all levels of the liquid vat and must be poured immediately to avoid any settling of ingredients. A vat with multiple pouring outlets at different heights would prevent any unevenness among films. In one embodiment, the edges of the films were sliced off from the mold before cutting into individual strips. The border of the mold could accumulate more solid ingredients and prevent quick dissolution in the user's oral cavity. Finally, the use of convective drying is highly recommended to reduce the drying time and prevent any degradation of active ingredients. Lowering the partial pressure of the vaporized solvent (ethanol) above the film casting plates will significantly speed up the evaporative process. Although the films will successfully dry over a 24-hour period of time at a safe temperature, this convective drying will allow for a considerably more efficient manufacturing process.

Although the present invention and some of its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim as follows:

1.-19. (canceled)

20. A consumable, bioadhesive film adapted to adhere to a inside of the oral cavity for the treatment of thermal burns or an injury comprising:
mucoadhesive layer to hold the film onto the cheek of the oral cavity;
a rate-controlling polymer layer to deliver a first and second active ingredient over an extended period of time;
wherein at least one active ingredient is embedded in the polymer layer;
a drug barrier layer to restrict the direction in which drug release can occur.
wherein the polymer disintegrates over time;
wherein the first active ingredient is for the treatment of pain; and
a second active ingredient for the healing of said thermal burn or injury.

21. The consumable film of claim 20 wherein the pharmaceutically active agent is selected from the group consisting of benzocaine, lidocaine, and menthol.

22. The consumable film of claim 20 wherein the first pharmaceutically active agent is benzocaine.

23. The consumable film of claim 22 wherein the benzocaine concentration is from about 0.1% to 20% by weight based on the total weight of the consumable film.

24. The consumable film of claim 23 wherein the benzocaine concentration is about 10% by weight based on the total weight of the consumable film.

25. The consumable film of claim 20 wherein the second pharmaceutically active agent is poloxamer 188.

26. The consumable film of claim 25 wherein the poloxamer 188 concentration is at least 15% by weight based on the total weight of the consumable film.

27. The consumable film of claim 26 wherein the benzocaine concentration is about 20% by weight based on the total weight of the consumable film.

28. The consumable film of claim 20 further having a sweetener that is selected from the group consisting of: monosaccharides, disaccharides and polysaccharides, soluble saccharin salts, L-aspartic acid derived sweetening agents, thaumatoccous danielli (Thaumatin I and II), sucralose, aspartame, acesulfame potassium, neotame, saccharin, xylitol.

29. The consumable film of claim 20 further including glycerol and HPMC.

30. The consumable film of claim 29 wherein the glycerol is present in the amount of about 10%.

31. The consumable film of claim 20 further including maltodextrin and sucralose.

32. The consumable film of claim 20 further including food grade coloring.

33. The consumable film of claim 20 further including an encasing selected from the group of pullulan, hydroxypropylmethyl cellulose (HPMC), hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

34. A method for delivering an agent for pain relief and healing within the oral cavity for the treatment of thermal burns or oral injuries comprising administering the consumable film of claim 1 to the oral cavity.

35. A method for producing a bioadhesive drug delivery film comprising:
by adding glycerol and slowly adding poloxamer 188 and stirring for approximately 3 to 5 minutes; and then adding benzocaine and heating and mixing at 60 degrees C. until the solution began to appeal clear or settled;
and then adding HPMC;
and then adding water in a drop-wise manner until the final solution was obtained.

36. The method of claim 35 further comprising adhering a drug barrier layer to a portion of the bioadhesive film.
